(19) United States
(12) Patent Application Publication
(10) Pub. No.: US 2017/0035598 A1
(43) Pub. Date: Feb. 9, 2017

(54) Integrated Cast And Limb Support System

(71) Applicant: Firefly Medical, Inc., Fort Collins, CO (US)

(72) Inventor: Stephen E. Schmutzer, Fort Collins, CO (US)

(73) Assignee: Firefly Medical, Inc., Fort Collins, CO (US)

(21) Appl. No.: 15/303,732

(22) PCT Filed: Apr. 21, 2015
(86) PCT No.: PCT/US15/26949

§ 371 Date: Oct. 12, 2016

Related U.S. Application Data
(60) Provisional application No. 61/982,757, filed on Apr. 22, 2014.

(51) Int. Cl. A61F 5/37; A61F 13/04; A61F 5/01

(52) U.S. Cl. CPC A61F 5/3738; A61F 5/0118; A61F 13/04

(57) ABSTRACT

A limb support coupler including a flexible body having opposing flexible body first and second faces, the flexible body conformingly embeddable within a limb support; and a connector outwardly extending from the flexible body first face. Further, a limb support coupler whereby the flexible body conformingly embeds within the limb support to dispose the flexible body first and seconds faces in adjacent generally parallel relation to respective limb support external and internal surfaces.

This International Patent Cooperation Treaty Patent Application claims the benefit of U.S. Provisional Patent Application No. 61/982,757, filed Apr. 22, 2014, hereby incorporated by reference herein.

### I. BACKGROUND

Conventional support structures may be employed following an injury to maintain an injured limb in a particular position. Often, a cast may be molded about the injured limb to secure the limb in the desired position. Additionally, a sling or other support structure may be utilized to support the cast and the injured limb within the cast. However, conventional support structures may not be configured to conform to the particular contours of the cast or correctly support the injured limb.

### II. DISCLOSURE OF THE INVENTION

Accordingly, a broad object of a particular embodiment of the invention can be to provide a limb support coupler including a flexible body having opposing flexible body first and second faces, the flexible body conformingly embeddable within a limb support; and a connector outwardly extending from the flexible body first face.

Another broad object of a particular embodiment of the invention can be to provide a limb support coupler whereby the flexible body conformingly embeds within the limb support to dispose the flexible body first and seconds faces in adjacent generally parallel relation to respective limb support external and internal surfaces.

Another broad object of a particular embodiment of the invention can be to provide a method of making a limb support coupler, the method including providing a flexible body having opposing flexible body first and second faces, whereby the flexible body is configured to conformingly embed within a limb support; and outwardly extending a connector from the flexible body first face.

Another broad object of a particular embodiment of the invention can be to provide a method of making a limb support coupler, the method further including configuring the flexible body to conformingly embed within the limb support to dispose the flexible body first and seconds faces in adjacent generally parallel relation to respective limb support external and internal surfaces.

Another broad object of a particular embodiment of the invention can be to provide a method of using a limb support coupler, the method including obtaining the limb support coupler with a flexible body having opposing flexible body first and second faces, the flexible body conformingly embeddable within a limb support, and a connector outwardly extending from the flexible body first face; and conformingly embedding the flexible body within the limb support.

Another broad object of a particular embodiment of the invention can be to provide a kit including at least one limb support coupler with a flexible body having opposing flexible body first and second faces, the flexible body conformingly embeddable within a limb support, and a connector outwardly extending from the flexible body first face; and a tether configured to couple to the connector.

Naturally, further objects of the invention are disclosed throughout other areas of the specification, drawings, and claims.

### III. BRIEF DESCRIPTION OF THE DRAWINGS

FIG. 1 is an illustration of a method of using a particular embodiment of a limb support coupler to support a limb support configured about a limb of a wearer.

FIG. 2A is a perspective view of a particular embodiment of a limb support coupler having protrusions outwardly extending from a flexible body first face of a flexible body of the limb support coupler.

FIG. 2B is a perspective view of a particular embodiment of a limb support coupler.

FIG. 3 is a side view of a particular embodiment of a limb support coupler.

FIG. 4 is an end view of a particular embodiment of a limb support coupler.

FIG. 5 is a top view of a particular embodiment of a limb support coupler.

FIG. 6 is a bottom view of a particular embodiment of a limb support coupler.

FIG. 7 is a top view of a particular embodiment of a plurality of limb support couplers provided as a flexible sheet having a plurality of connectors outwardly extending from a flexible sheet first face.

FIG. 8A is a perspective view of a particular embodiment of a securement element configured to couple to a limb support coupler.

FIG. 8B is a perspective view of a particular embodiment of a limb support coupler embedded within a limb support, a securement element coupled to the limb support coupler, and a tether coupled to the securement element.

FIG. 9 is an illustration of a method of making a particular embodiment of a limb support coupler.

FIG. 10 is an illustration of a method of making a particular embodiment of a one-piece construct including a limb support and a limb support coupler.

FIG. 11A is a perspective view of a particular embodiment of a limb support coupler embedded within a limb support.

FIG. 11B is a cross-sectional view 11B-11B of the particular embodiment of the limb support coupler embedded within the limb support shown in FIG. 11A.

### IV. MODE(S) FOR CARRYING OUT THE INVENTION

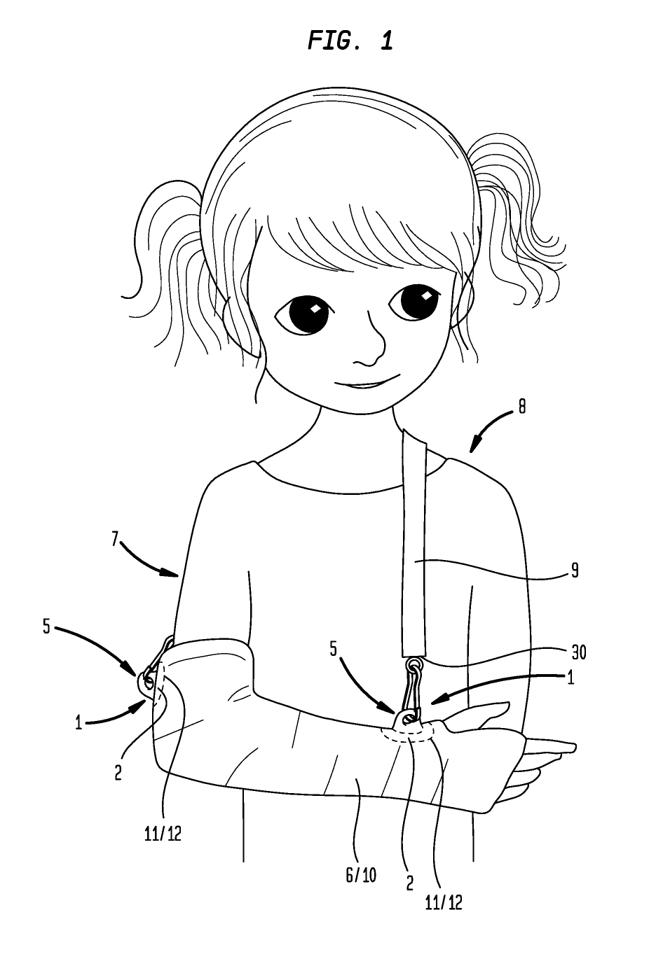

Now referring primarily to FIG. 1, which illustrates a method of using a particular embodiment of a limb support coupler (1) including a flexible body (2) having opposing flexible body first and second faces (3)(4) and a connector (5) outwardly extending from the flexible body first face (3), whereby the method of use can include conformingly embedding the flexible body (2) within a limb support (6) configured about a limb (7) of a wearer (8). As to particular embodiments, the method of using the limb support coupler (1) can, but need not necessarily, further include coupling a tether (9) to the connector (5), whereby the tether (9) can be coupled about the wearer (8) of the limb support (6), for example about an upper portion of the wearer's body as shown in the example of FIG. 1, to support the limb support (6) and correspondingly, to support the limb (7).

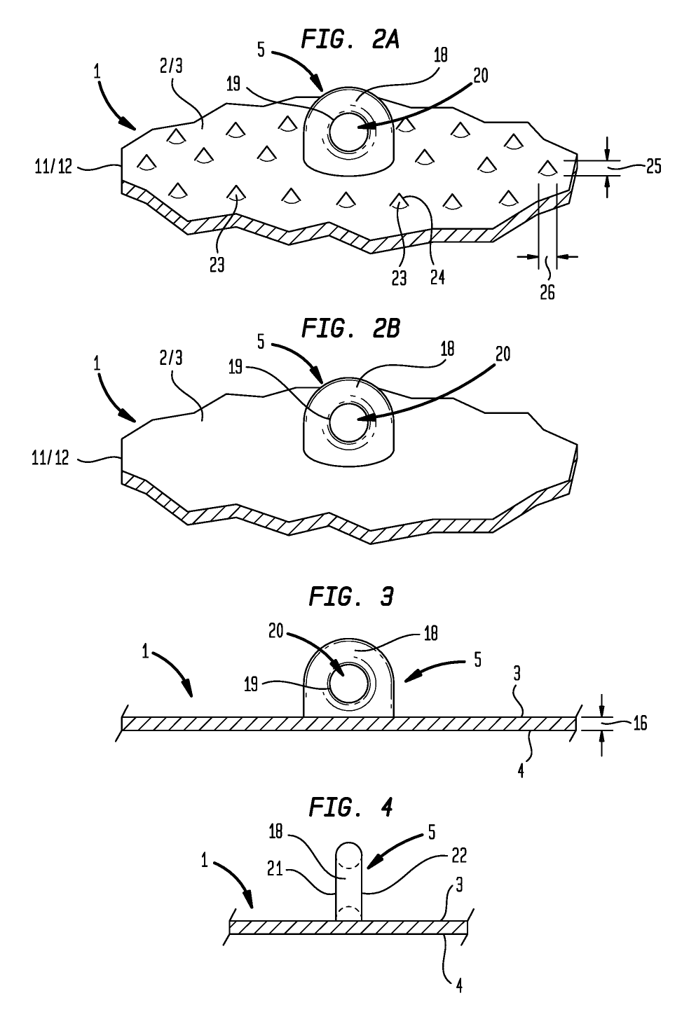

Now referring primarily to FIG. 2A through FIG. 6, the limb support coupler (1) includes a flexible body (2) having opposing flexible body first and second faces (3)(4), whereby the flexible body (2) is conformingly embeddable within a limb support (6) or is configured to conformingly embed within a limb support (6). Further, the limb support coupler (1) includes a connector (5) outwardly extending from the flexible body first face (3) and correspondingly, from a limb support external surface (10) when the flexible body (2) is embedded within a limb support (6).

Again referring primarily to FIG. 2A through FIG. 6, the flexible body first and second faces (3)(4), which can be substantially planar (as shown in the examples of the Figures) or arcuate, can extend to a flexible body outer edge (11) which defines a flexible body periphery (12).

The flexible body periphery (12) can define any of a numerous and wide variety of configurations of varying dimensions, including or consisting of: a circle; an oval; an ellipse; a triangle; a square; a rectangle; a trapezoid; a polygon; a freeform configuration; an image, for example a human image, an animal image, or a fanciful creature image; or the like, or combinations thereof, depending upon the application.

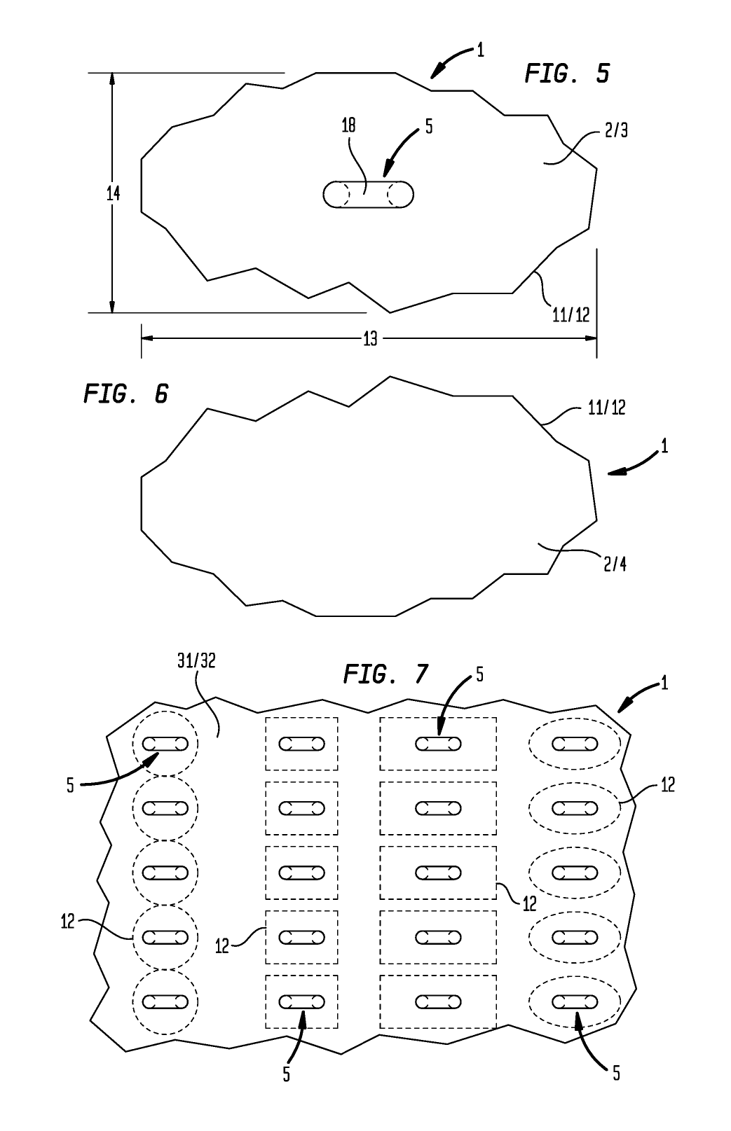

As but one illustrative example, the flexible body (2) can include a flexible body outer edge (11) which defines a generally elliptical flexible body periphery (12) (as shown in the example of FIG. 1 in broken line), whereby the dimensional relations of the generally elliptical flexible body periphery (12) can include a flexible body length (13) (as shown in the example of FIG. 5) of about 3 inches and a flexible body width (14) (as also shown in the example of FIG. 5) of about 1.5 inches.

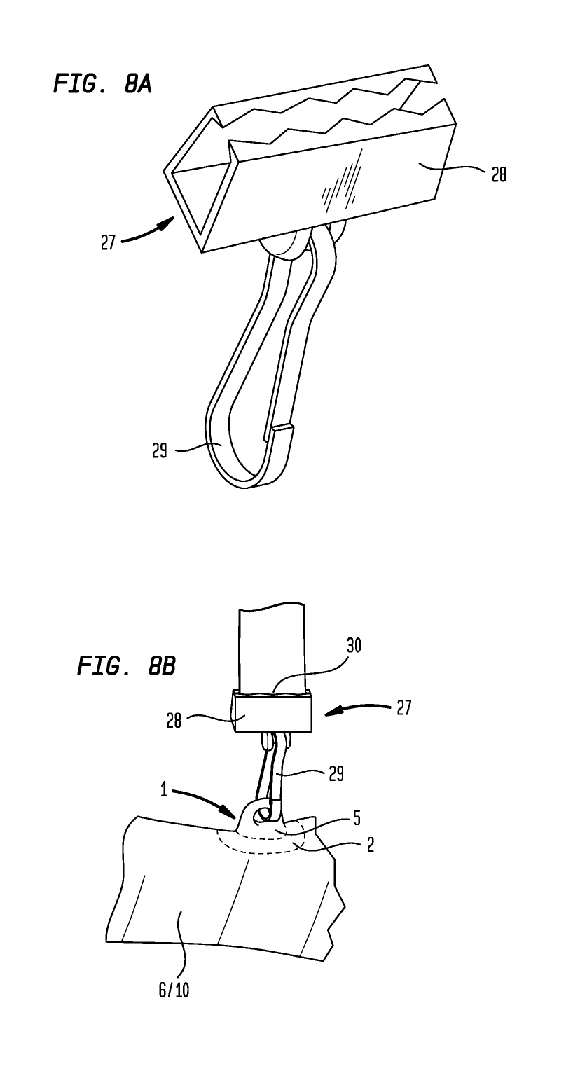

Now referring primarily to FIG. 7, as to particular embodiments, the flexible body periphery (12) can be customizable to provide a customized flexible body periphery (12) which can be specifically suited for a particular application. As but one illustrative example, the flexible body periphery (12) can be cuttable or relatively easily cut such that upon cutting, the customized flexible body periphery (12) can be generated.

As to particular embodiments of the limb support coupler (1) having a cuttable flexible body periphery (12), the cuttable flexible body periphery (12) can be cut to generate the customized flexible body periphery (12) by any of a wide variety of processes using any of a wide variety of cutting apparatuses including or consisting of: cutting machines, cutting devices, knives, blades, scalpels, scissors, shears, lasers, water jets, or the like, or combinations thereof, depending upon the application.

Now referring primarily to FIG. 2A through FIG. 4, the flexible body (2) can include a flexible body thickness (16) (as shown in the example of FIG. 3) disposed between the flexible body first and second faces (3)(4), whereby the flexible body thickness (16) can be either substantially uniform or non-uniform between the flexible body periphery (12). As an illustrative example of the latter, the flexible body (2) can include a flexible body thickness (16) which can be greater proximate a central portion of the flexible body (2) relative to a peripheral portion of the flexible body (2). As another illustrative example, the flexible body (2) can include a flexible body thickness (16) which can be lesser proximate a central portion of the flexible body (2) relative to a peripheral portion of the flexible body (2).

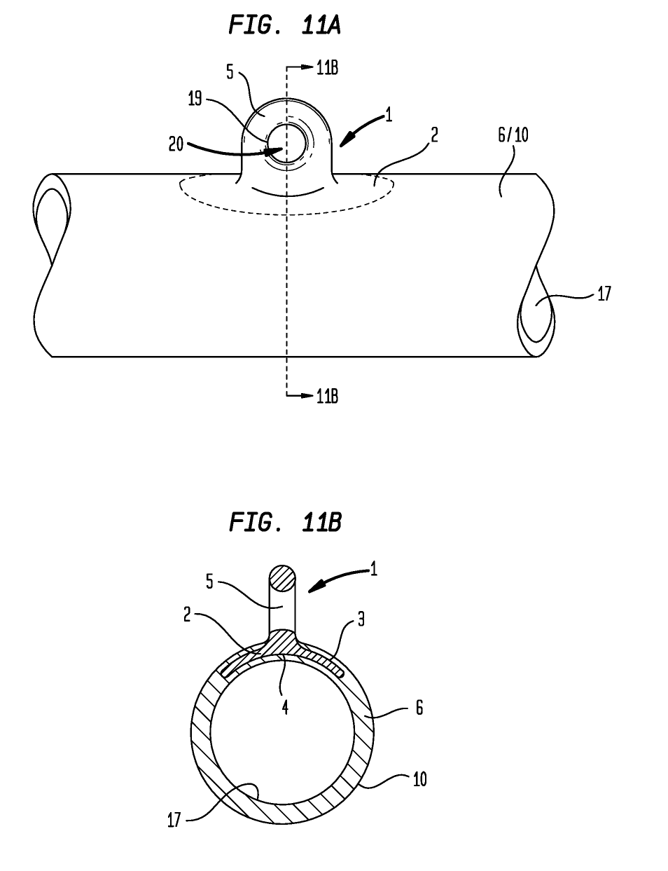

Now referring primarily to FIG. 11A and FIG. 11B, the flexible body (2) is conformingly embeddable within a limb support (6) or is configured to conformingly embed within a limb support (6). As to particular embodiments, the flexible body (2) can conformingly embed within a limb support (6) to dispose the flexible body first and seconds faces (3)(4) in adjacent generally parallel relation to respective limb support external and internal surfaces (10)(17) (as shown in the example of FIG. 11B).

The conformingly embeddable property of the flexible body (2) may be particularly useful when embedding the flexible body (2) within a portion of a limb support (6) which is nonplanar or arcuate. As to particular embodiments, the nonplanar or arcuate portion of the limb support (6) can conform about a nonplanar or arcuate portion of a limb (7) about which it configures and supports. As most limbs (7) requiring support by a limb support (6) have nonplanar or arcuate portions, there is a substantial advantage to a limb support coupler (1) having a flexible body (2) which can conformingly embed within a nonplanar or arcuate portion of a limb support (6).

Now referring primarily to FIG. 2 through FIG. 5, the limb support coupler (1) further includes a connector (5) outwardly extending from the flexible body first face (3) and correspondingly, from a limb support external surface (10) when the flexible body (2) is embedded within a limb support (6).

As to particular embodiments, the connector (5) can, but need not necessarily, include a connector body (18) which can have any of a numerous and wide variety of configurations of varying dimensions suitable for outwardly extending from the flexible body first face (3) and coupling the flexible body (2) conformingly embedded within a limb support (6) with a tether (9) configured to couple about a wearer (8) of the limb support (6) to support the limb support (6) and correspondingly, to support the limb (7). As non-limiting examples, the connector body (18) can be configured as a loop, an eye, an eyelet, a hook, or the like, or combinations thereof.

Again referring primarily to FIG. 2 through FIG. 5, the connector body (18) can include an aperture element (19) defining an aperture element opening (20) which communicates between connector body first and second sides (21)(22). As but one illustrative example, the aperture element (19) can have a substantially circular configuration which defines a substantially circular aperture element opening (20); however the invention need not be so limited, as the aperture element (19) and correspondingly, the aperture element opening (20), can have any of a numerous and wide variety of configurations of varying dimensions, depending upon the application.

The connector (5) outwardly extending from the flexible body first face (3) can be disposed in numerous and varied locations on the flexible body first face (4). While the illustrative example of the connector (5) depicted in the Figures shows the connector (5) disposed in a generally central location on the flexible body first face (3) (as shown in the example of FIG. 5), embodiments of the limb support coupler (1) can have a connector (5) disposed in any suitable location on the flexible body first face (3), including non-central or peripheral locations, whereby the location permits a tether (9) to be coupled to the connector (5) and correspondingly, to the flexible body (2) embedded within a limb support (6). Further, the connector (5) can be disposed in any location on the flexible body first face (4) which precludes disengagement from the tether (9) or the limb support (6) in which the flexible body (2) is embedded. Still further, the connector (5) can be disposed in any location on the flexible body first face (3) which minimizes forces directed to the discrete injury site within the limb (7) supported by the limb support (6).

As to particular embodiments, the limb support coupler (1) can include a plurality of connectors (5) outwardly extending from the flexible body first face (3), whereby the connectors (5) can have connector bodies (18) which have similar or dissimilar configurations, dimensions, or combinations thereof, depending upon the application.

Now referring primarily to FIG. 2A, as to particular embodiments, the flexible body (2) can, but need not necessarily, further include at least one protrusion (23) outwardly extending from the flexible body first face (3), the flexible body second face (4), or combinations thereof, whereby the one or more protrusions (23) can function to further engage the flexible body first and second faces (3)(4) with the limb support (6) when the flexible body (2) is embedded within the limb support (6) to assist in maintaining the position of the flexible body (2) within the limb support (6).

Again referring primarily to FIG. 2A, the protrusion (23) can include a protrusion periphery (24) which can define any of a numerous and wide variety of configurations of varying dimensions, depending upon the application. While the illustrative example of the protrusion periphery (24) shown in FIG. 2A defines a substantially conical configuration, embodiments can have a protrusion periphery (24) defining any suitable three-dimensional configuration, including or consisting of: a cone, a cube, a cylinder, a domed cylinder, an ellipsoid, a prism, a pyramid, a trapezoid, a sphere, or the like, or combinations thereof, depending upon the application, which allows the protrusion (23) to assist in maintaining the position of the flexible body (2) within the limb support (6).

Yet again referring primarily to FIG. 2A, as but one illustrative example, a protrusion (23) can include a protrusion periphery (24) defining a cone, whereby a protrusion length (25) can be about ⅛ inch and a protrusion width (26) can be about ¼ inch.

Now referring primarily to FIG. 8A and FIG. 8B, as to particular embodiments, the limb support coupler (1) can, but need not necessarily, further include one or more securement elements (27) coupled or removably coupled to the connector (5), whereby the securement element (27) can be configured to couple a tether (9) to the connector (5).

The securement element (27) can have any of a numerous and wide variety of configurations of varying dimensions, depending upon the application, including adhesive elements, mechanical fastening elements, or the like, or combinations thereof. Exemplary adhesive elements can include, as non-limiting examples: non-reactive adhesives including drying adhesives, pressure-sensitive adhesives, contact adhesives, or hot adhesives; reactive adhesives including one-part adhesives or multi-part adhesives; natural adhesives; synthetic adhesives; or the like, or combinations thereof. Exemplary mechanical fasteners can include, as non-limiting examples: buckles, buttons, clamps, clips, grommets, hook-and-eye closures, mated hook and loop fasteners, pins, rivets, snap fasteners, staples, stitches, straps, tape, zippers, or the like, or combinations thereof.

Again referring primarily to FIG. 8A and FIG. 8B, as but on illustrative example, the securement element (27) can be configured as a clamp element (28) which, as to particular embodiments, can couple to a linking element (29). The clamp element (28) can be clamped about a tether end (30) to couple the securement element (27) to the tether (9) and the linking element (29) can be coupled to the connector (5).

A method of making a particular embodiment of a limb support coupler (1) can including providing a flexible body (2) having opposing flexible body first and second faces (3)(4), whereby the flexible body (2) is configured to conformingly embed within a limb support (6); and outwardly extending a connector (5) from the flexible body first face (3).

As to particular embodiments, the method of making the limb support coupler (1) can, but need not necessarily, further include configuring the flexible body (2) to conformingly embed within a limb support (6) to dispose the flexible body first and seconds faces (3)(4) in adjacent generally parallel relation to respective limb support external and internal surfaces (10)(17).

As to particular embodiments, the method of making the limb support coupler (1) can, but need not necessarily, further include configuring a flexible body periphery (12), which is defined by a flexible body outer edge (11) to which the flexible body first and second faces (3)(4) extend, as a customizable flexible body periphery (12).

As to particular embodiments, the method of making the limb support coupler (1) can, but need not necessarily, further include configuring the customizable flexible body periphery (12) as a cuttable flexible body periphery (12) whereby, upon cutting, a customized flexible body periphery (12) can be generated.

As to particular embodiments, the method of making the limb support coupler (1) can, but need not necessarily, further include disposing an aperture element (19) in a connector body (18) of the connector (5), the aperture element (19) defining an aperture element opening (20) which communicates between connector body first and second sides (21)(22).

As to particular embodiments, the method of making the limb support coupler (1) can, but need not necessarily, further include outwardly extending a plurality of connectors (5) from the flexible body first face (3).

As to particular embodiments, the method of making the limb support coupler (1) can, but need not necessarily, further include outwardly extending at least one protrusion (23) from the flexible body first face (3), the flexible body second face (4), or combinations thereof.

As to particular embodiments, the method of making the limb support coupler (1) can, but need not necessarily, further include coupling or removably coupling a securement element (27) to the connector (5), the securement element (27) configured to couple a tether (9) to the connector (5).

As to particular embodiments, the method of making the limb support coupler (1) can, but need not necessarily, further include configuring the securement element (27) as a clamp element (28).

Components of the limb support coupler (1) can be entirely formed of the same material, or alternatively, components of the limb support coupler (1) can be formed from different materials, including any of a numerous and wide variety of flexible materials, resiliently flexible materials, or the like, or combinations thereof. By way of non-limiting example, the material can be soft, breathable, moisture-wicking, anti-bacterial, natural, synthetic, odor-resistant, or machine washable and can include or consist of: cotton, linen, rayon, viscose, ramie, hemp, jute, leather, leather-like material, rubber, rubber-like material, plastic, plastic-like material, acrylic, polyamide, polyester, microfiber, polypropylene, polyvinyl chloride-based materials, silicone-based materials, or the like, or combinations thereof.

The limb support coupler (1) or components of the limb support coupler (1) can be produced from any of a numerous and wide variety of processes, such as press molding, injection molding, fabrication, machining, printing, additive printing, or the like, or combinations thereof, as a one-piece construct or provided as a plurality of pieces for assembly into an embodiment of the limb support coupler (1).

Now referring primarily to FIG. 7, as to particular embodiments, a plurality of limb support couplers (1) can be provided as a flexible sheet (31) having a plurality of connectors (5) outwardly extending from a flexible sheet first face (32). The flexible sheet (31) can be cuttable or relatively easily cut around each connector (5) to generate a plurality of limb support couplers (1), each having a customized flexible body periphery (12).

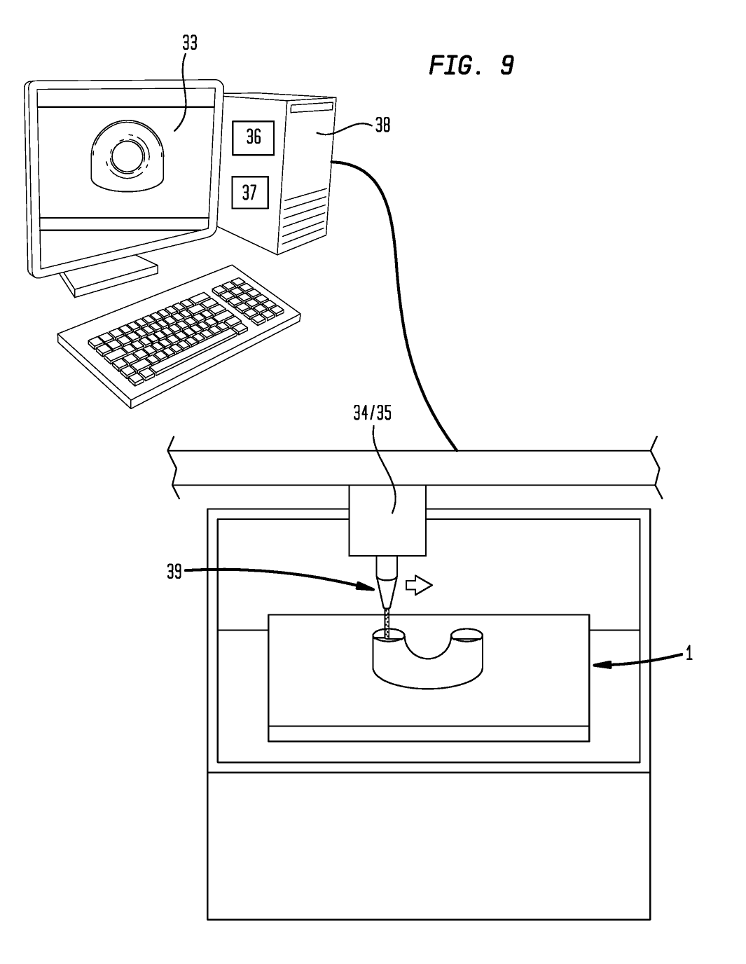

Now referring primarily to FIG. 9, the limb support coupler (1) can be produced from a digital model (33) providing a data representation of the limb support coupler (1) which can be rendered in tangible form by operation of a three-dimensional materials deposition apparatus (34) or materials printer (35).

As to particular embodiments, the digital model (33) can be produced by Computer Aided Design (CAD) software applications or three-dimensional scanning systems, both of which are available from numerous different suppliers. The digital model (33) can be stored in industry-standard file formats as a digital model file (36), which can be transmitted electronically and interpreted by a three-dimensional materials deposition application program (37) or a materials printer program (37) (also referred to as an “application program”) to provide computer program instructions which can be loaded onto a computer (38) to implement functions necessary to control the three-dimensional materials deposition apparatus (34) or materials printer (35) and the additive build-up process (39) to produce the limb support coupler (1) represented by the digital model (33). The computer (38) can take the form of a general purpose computer, special purpose computer, or other programmable data processing apparatus.

As to particular embodiments, the computer (38) can run the application program (37) which reads the digital model file (36), accepts parameter and preference inputs from a user, performs a series of detailed calculations, and transmits commands to the three-dimensional materials deposition apparatus (34) or materials printer (35) (which as to particular embodiments can be a three-dimensional printer such as a ZPRINTER® 350 available from Z Corporation or a three-dimensional printer assembly as described in U.S. Pat. No. 7,037,382 which is incorporated by reference herein, or similar apparatus), thereby providing information necessary for fabricating the desired three-dimensional limb support coupler (1).

As to particular embodiments, the three-dimensional materials deposition apparatus (34) or materials printer (35) can comprise a three-dimensional printer having an array of jet-type print heads which deposit conventional binder liquid onto successive layers of a conventional build material, for example, as disclosed in U.S. Pat. No. 5,902,441, which is incorporated by reference herein. Where the conventional binder liquid combines with the conventional build material, the conventional build material can react and harden. By controlling the placement of binder droplets from these print heads, the solid structure of the three-dimensional limb support coupler (1) can be physically reproduced by an additive build-up process (39). Further details of binding a conventional build material with a conventional binder liquid to form an object are disclosed in U.S. Pat. No. 5,340,656 to Sachs et al. and U.S. Pat. No. 5,387,380 to Cima et al., which are incorporated by reference herein.

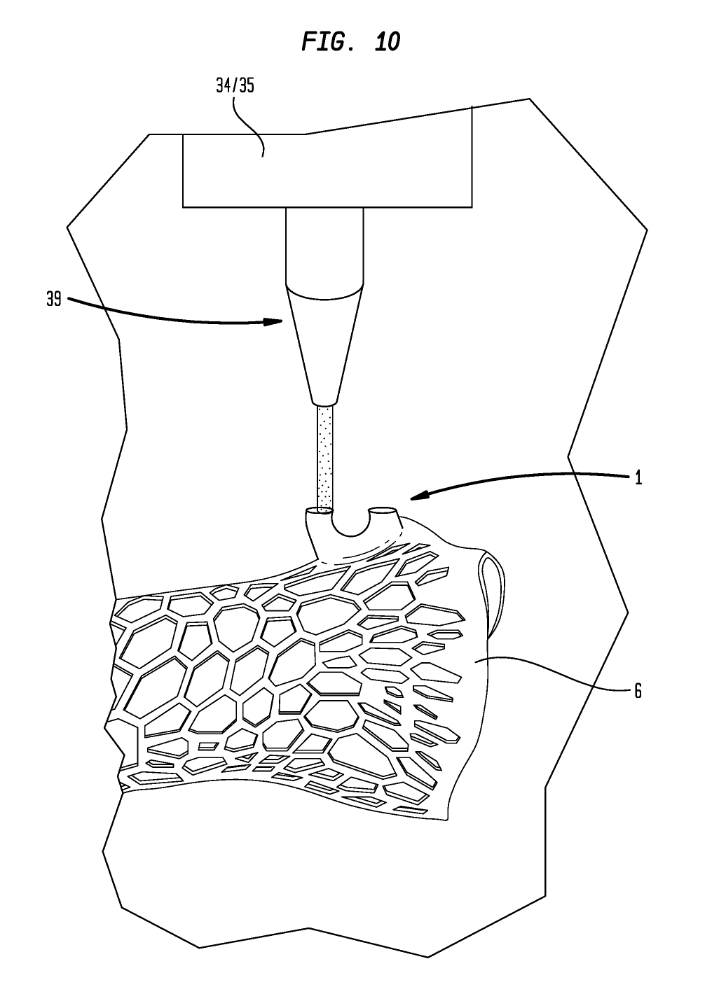

Now referring primarily to FIG. 10, the digital model (33) above described can provide a data representation of the limb support (6) including the limb support coupler (1) to be rendered as a unitary construct in tangible form by operation of the three-dimensional materials deposition apparatus (34) or materials printer (35). As an illustrative example, a limb (7) can be three-dimensionally scanned using medical imaging methodologies, including as illustrative examples, techniques such as radiology, which can use imaging technologies including X-ray radiography, magnetic resonance imaging, medical ultrasonography, ultrasound, endoscopy, elastography, tactile imaging, thermography, medical photography, nuclear medicine functional imaging techniques, or the like, or combinations thereof. Based on the three-dimensional scan of the limb (7), a digital model (33) of the limb support (6) can be generated having a configuration suitable to support the limb (7) to promote healing of a discrete injury site within the limb (7).

The limb support (6) can be produced by operation of the three-dimensional materials deposition apparatus (34) or materials printer (35) as above described. The digital model (33) can further include the limb support coupler (1) which can be produced as a one-piece construct with the limb support (6). The location of the limb support coupler (1) within the digital model (33) can be pre-determined such that the connector (5) can outwardly extend from the limb support (6) at a location which allows forces applied by a tether (9) upon the limb support (6) to be minimized at the discrete injury site within the limb (7).

Again referring primarily to FIG. 10, as to particular embodiments, the one-piece construct including the limb support (6) and the limb support coupler (1) can be configured as an exoskeleton with a trabecular bone structure, a honeycomb-like structure, or the like, or combinations thereof.

A method of using a particular embodiment of a limb support coupler (1) can include obtaining the limb support coupler (1) and conformingly embedding a flexible body (2) of the limb support coupler (1) within a limb support (6).

The flexible body (2) can be conformingly embedded within a limb support (6) formed from any of a numerous and wide variety of materials suitable for encasing a limb (7), including moldable materials such as plaster, fiberglass, plastic, paper, printing materials, additive printing materials, or the like, or combinations thereof.

While the limb support (6) is being cast about a limb ( )7) of a wearer (8), the flexible body (2) of the limb support coupler (1) can be conformingly embedded within the limb support (6), for example by wrapping a portion of the limb support (6) about the flexible body (2) of the limb support coupler (1) while wrapping a portion of the limb support (6) about the limb (7) of the wearer (8).

The method of using the limb support coupler (1) can, but need not necessarily, further include coupling a tether (9) to the connector (5).

The method of using the limb support coupler (1) can, but need not necessarily, further include coupling the tether (9) about a wearer (8) of the limb support (6).

The method of using the limb support coupler (1) can, but need not necessarily, further include obtaining a pair of limb support couplers (1) and conformingly embedding the flexible body (2) of each limb support coupler (1) within the limb support (6).

The method of using the limb support coupler (1) can, but need not necessarily, further include coupling one of a pair of opposing tether ends (30) of a tether (9) to each connector (5) of each limb support coupler (1). As to particular embodiments, the method of using the limb support coupler (1) can, but need not necessarily, further include coupling the tether (9) about a wearer (8) of the limb support (6).

The limb support coupler (1) can be provided as a kit including at least one limb support coupler (1) comprising a flexible body (2) having opposing flexible body first and second faces (3)(4), the flexible body (2) conformingly embeddable within a limb support (1), and a connector (5) outwardly extending from the flexible body first face (3); and a tether (9) configured to couple to the connector (5).

The limb support coupler (1) can be provided as a kit which can, but need not necessarily, further include a securement element (27) configured to couple the tether (9) to the connector (5). As to particular embodiments, the securement element (27) can be configured as a clamp element (28).

The limb support coupler (1) can be provided as a kit wherein the flexible body first and second faces (3)(4) can, but need not necessarily, extend to a flexible body outer edge (11) which defines a flexible body periphery (12), the flexible body periphery (12) customizable to provide a customized flexible body periphery (12). As to particular embodiments, the flexible body periphery (12) can be cuttable or relatively easily cut such that upon cutting, the customized flexible body periphery (12) can be generated.

The limb support coupler (1) can be provided as a kit which can, but need not necessarily, further include a cutting apparatus configured to cut the flexible body periphery (12). As to particular embodiments, the cutting apparatus can be selected from the group including or consisting of: cutting machines, cutting devices, knives, blades, scalpels, scissors, shears, lasers, water jets, or the like, or combinations thereof.

As can be easily understood from the foregoing, the basic concepts of the present invention may be embodied in a variety of ways. The invention involves numerous and varied embodiments of an integrated cast and limb support system and methods for making and using such integrated cast and limb support systems, including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “support” should be understood to encompass disclosure of the act of “supporting”—whether explicitly discussed or not—and, conversely, were there effectively disclosure of the act of “supporting”, such a disclosure should be understood to encompass disclosure of a “support” and even a “means for supporting”. Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the integrated cast and limb support systems herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A limb support coupler comprising:
a flexible body conformingly embeddable within a limb support, said flexible body having opposing flexible body first and second faces which are substantially planar; and
only one connector outwardly extending from said flexible body first face.

2. The limb support coupler of claim 1, wherein said flexible body conformingly embeds within said limb support to dispose said flexible body first and seconds faces in adjacent generally parallel relation to respective limb support external and internal surfaces.

3. The limb support coupler of claim 2, wherein said flexible body first and second faces extend to a flexible body outer edge which defines a flexible body periphery, said flexible body periphery customizable to provide a customized flexible body periphery.

4. The limb support coupler of claim 3, wherein said flexible body periphery is cuttable such that upon cutting, said customized flexible body periphery is generated.

5. The limb support coupler of claim 2, wherein said connector comprises a connector body having an aperture element defining an aperture element opening which communicates between connector body first and second sides.

6. (canceled)

7. The limb support coupler of claim 5, wherein said flexible body further comprises at least one protrusion outwardly extending from said flexible body first face, said flexible body second face, or combinations thereof.

8. The limb support coupler of claim 5, further comprising a securement element coupled to said connector, said securement element configured to couple a tether to said connector.

9. The limb support coupler of claim 8, wherein said securement element is configured as a clamp element.

10-25. (canceled)

26. A kit comprising:
at least one limb support coupler comprising a flexible body conformingly embeddable within a limb support, said flexible body having opposing flexible body first and second faces which are substantially planar;
only one connector outwardly extending from said flexible body first face; and
a tether configured to couple to said connector.

27. The kit of claim 26, further comprising a securement element configured to couple said tether to said connector.

28. The kit of claim 27, wherein said securement element is configured as a clamp element.

29. The kit of claim 26, wherein said flexible body first and second faces extend to a flexible body outer edge which defines a flexible body periphery, said flexible body periphery customizable to provide a customized flexible body periphery.

30. The kit of claim 29, wherein said flexible body periphery is cuttable such that upon cutting, said customized flexible body periphery is generated.

31. The kit of claim 30, further comprising a cutting apparatus configured to cut said flexible body periphery.

32. The kit of claim 31, wherein said cutting apparatus is selected from the group consisting of: cutting machines, cutting devices, knives, blades, scalpels, scissors, shears, lasers, water jets, and combinations thereof.
